(19) United States
(12) Patent Application Publication
(10) Pub. No.: US 2011/0097338 A1
(43) Pub. Date: Apr. 28, 2011

(54) Use of Substances for Sensitization of Tumor Cells to Radiation and/or Chemotherapy

(75) Inventors: Nils Cordes, Dresden (DE); Michael Baumann, Dresden (DE); Iris Eke, Dresden (DE); Ulrike Koch, Dresden (DE); Veit Sandfort, Berlin (DE)

(73) Assignee: TECHNISCHE UNIVERSITAET DRESDEN, Dresden (DE)

(21) Appl. No.: 12/918,496

(22) PCT Filed: Feb. 20, 2009
(86) PCT No.: PCT/EP09/52058

§ 371 Date: Dec. 10, 2010

(30) Foreign Application Priority Data
Feb. 20, 2008 (DE) 10 2008 011 838.9

(51) Int. Cl. A61K 39/395; C12N 5/09; A61P 43/00

(52) U.S. Cl. 424/156.1; 435/375; 424/174.1

(57) ABSTRACT

The invention relates to the use of substances to increase the sensitivity of tumor cells to treatment with radiation and/or chemotherapy. This is accomplished through the use of substances which block or limit the function of the PINCH-1 protein for sensitization of tumor cells to radiation and/or chemotherapy.

The invention concerns the use of substances for increasing the sensitivity of tumor cells to treatment with radiation and/or chemotherapy.

The killing of human cancer cells by radiation and/or cytostatic agents (chemotherapy) belongs to the most important treatment methods in the fight against cancer. However, it is not possible to this day to damage entirely specifically the tumor tissue, so that the treatment also affects healthy tissue and thereby serious side effects of the treatment occur and decrease the chances for curing. Hence, there is a need for treatment strategies that enable to carry out the treatment of the patient as individually and briefly as possible in order to keep the side effects as minimal as possible. This is enabled in particular by modern radiotherapy that kills efficiently the most important cell population within a tumor, the tumor stem cells, and prevents therefore a regrowth after treatment (tumor recidivism). Nevertheless, under certain clinical circumstances, the application of such a radiation dose is not possible so that at the same time chemotherapy and/or new molecular therapies are used in order to eradicate the tumor stem cells. Preclinical in vitro and in vivo examinations, as further shown below, demonstrate whether a molecular therapy against a specific molecule removes the tumor stem cells alone and/or in combination with radiotherapy. If this is the case, the patient is cured and there is no tumor recidivism. Another determining criterion for the use of a specific molecular therapy is the knowledge about the presence of the target molecule in the tumor tissue. These examinations are carried out on human tumor biopsies and show whether the molecule in question is expressed or overexpressed.

The object of the present invention is to make the tumor cells more sensitive for the treatment by radiation and/or chemotherapy in order to reduce the duration and strength of the radiation and/or chemotherapy and thus also the side effects associated with the radiation and/or chemotherapy.

According to the invention, the object is solved by administering substances that block or limit the function of the PINCH-1 protein. The PINCH-1 protein is called in the literature sometimes also by the name LIMS1 or “LIM and senescent cell antigen like domains 1”. According to the invention the inventive substances are administered to tumor cells. The term tumor cells in the context of the invention encompasses all tumor cells that may appear in connection with any kind of malignant tumor disease in hematologic as well as solid tumors. Within the context of the present invention it has been surprisingly found that blocking of the PINCH-1 protein (functionally or at the expression level) leads in tumor cells to an increase of the sensitivity relative to radiation or chemotherapy. After the treatment with radiation and/or cytostatic agents (chemotherapy) the tumor cells show a clearly reduced survival rate compared to tumor cells in which the PINCH-1 protein had not been blocked.

For chemotherapy, for example, the substances cisplatin or mitomycin C, but also all the other conventional chemotherapies, according to the tumor entity to be treated, can be used. Chemotherapeutic agents are preferably selected from 5′-deoxy-5-fluorouridin, 5-fluorouracil, 6-mercaptopurine, 6-thioguanine, 9-aminocamptothecin, abarelix, azacitidine, actinomycin D, aldesleukin, alemtuzumab (MabCampath®), alitretinoin, altretamin, ametantron, amifostine, aminoglutethimide, amsacrine, anagrelide, anastrozole, arsenic (III) oxide, asparaginase, atrasentan (Xinlay®), azathioprine, BCG live (Theracys), bendamustine, bevaceizumab, bexarotene, bicalutamide, biolimus A9, bleomycin, bortezomib, buserelin, busulfan, calicheamicin, calusteron, camptothecine, capecitabine, carboplatin, carmustine, cetuximab, chlorambucil, chlorethamin, cinacalcet, cisplatin, cladribine, cyclophosphamide, cyproterone acetate, cytarabine, cytosinarabinoside, dacarbazine, dactinomycin, darbepoetin alpha, dasatinib (Sprycel®), daunorubicin, denileukin diftitox, dexrazoxane, docetaxel, doxorubicin (adriamycin), dromostanolone, Elliott's B solution, epirubicin (4-epi adriamycin), erlotinib (Tarceva®), erythropoietin, estramustine, etoposide, everolimus (Certicane®), exemestane, filgrastim, floxuridine, fludarabine, fluorouracil, flutamide, formestane, fosfestrol, fulvestrant, G-CSF, gefitinib (Iressa®), gemcitabine, gemtuzumab ozogamicin, goserelin, hydroxycarbamide (hydroxyurea), ibritumomab (Zevalin®), idarubicin, ifosfamide, imatinib, interferon alpha, irinotecan, ixabepilone, lanreotide, lapatinib (Tykerb®), lenalidomide (Revlimide), letrozole, leucovorin, leuprorelin, levamisole, lonafamib (Sarasar®), interleukin-2, lomustine, maytansinoid, meclorethamin, megestrol, melphalan, mercaptopurine, mesna, methotrexate, methoxsalene, methylprednisolone, miltefosine, mitomycin C, mitopodozide, mitotane, mitoxantrone, nandrolone, nelarabine, nilotinib (Tasigna®), nimustine, nofetumomab, oblimersen, octreotide, oprelvekin, oxaliplatin, oxazaphosphorine, paclitaxel and paclitaxel derivates, pamidronate, panitumumab, pegademase, pegaspargase, pegfilgrastim, pemetrexed, pentostatin, pipobroman, plicamycin, podophyllotoxin derivates, polifeprosan, porfimer, prednisone, procarbazine, quinacrine, raltitrexed, rapamycin (sirolimus), rasburicase, retinol, rhodomycin D, rituximab (MabThera®), sargramostim, sorafenib (Nexavar®), streptozocin, sunitinib (Sutent®), tamoxifen, tegafur, temozolomide, temsirolimus, teniposide, testolactone, thalidomide, thioguanine, thiotepa, tipifamid (Zarnestra®), topotecan (topoisomerase I inhibitor), toremifene, tositumomab, trabectedine, trastuzumab (Herceptin®), treosulfan, tretinoin, triptorelin, trofosfamide, uramustin, valrubicin, vinorelbine, vinblastine, vincristine, vindesine, vinorelbine and zoledronate.

As a radiation type the radiation with photons (X-rays, y radiation), electrons, protons or heavy ions (1-H, 2-hey, 7-Li, 9-Be, 11-B, 12-C, 14-N or 16-O) is preferably used.

The invention encompasses the use of substances for the sensitization of tumor cells against radiation and/or chemotherapy in that the function of the PINCH-1 protein at the level of the genetic expression or at the protein level is blocked or is limited.

The invention encompasses furthermore a method for treating a tumor patient with which method the tumor cells are first sensitized first by administering substances that block or limit the function of the PINCH-1 protein at the level of gene expression or at the protein level, relative to radiation and/or chemotherapy and are treated subsequently by radiation and/or chemotherapy. The method for the treatment of a benign or malignant tumor in a living being encompasses the following steps:
a) one or several substances that block or limit the function of the PINCH-1 protein at the level of the gene expression or at the protein level are administered to a living being having a tumor topically or systemically by which substance the tumor is sensitized relative to the radiation therapy and/or chemotherapy performed in step b).
b) Afterwards the tumor is treated by radiation therapy and/or chemotherapy.

In this connection, the living being can be an animal or a human being. Preferably, the inventive method is suitable for the use in a human being.

Within the context of the present invention, the substances which block or limit the function of the PINCH-1 protein are to be understood in accordance with the broadest possible meaning. Thus, substances are included that inhibit the PINCH-1 protein at the protein level by direct binding as, for example, antibodies or natural or modified ligands, as well as substances that prevent or reduce significantly the expression of the gene at the transcriptional or post-transcriptional level and effect in this way that no PINCH-1 protein is formed at all or not enough, for example, RNA interference or transcription factors.

The use of the inventive substances is suited preferably for all kinds of tumors in which the PINCH-1 protein exists, preferably for such tumors in which the PINCH-1 protein is overexpressed.

According to the invention, as substances that block or limit the function of the PINCH-1 protein, such agents are considered that are suitable to reduce, suppress or prevent the function of the PINCH-1 protein at the level of the gene expression or the protein level. They encompass inter alia organo-chemical compounds, peptide analogs, peptide mimicking agents, nucleic acids, oligo- and poly-nucleotides, antibodies etc. The substances can be applied either directly as an active agent, or be formed as so-called “prodrugs” by the endogenic metabolism.

As inventive substances that block or limit the function of the PINCH-1 protein, preferably anti-PINCH-1-antibodies, PINCH-1-dsRNA, PINCH-1-siRNA, PINCH-1-short hairpin (sh)RNA and PINCH-1-morpholinos (also called morpholino oligos, phosphorodiamidate morpholino oligos or PMOs) are used.

In one embodiment of the invention, anti-PINCH-1-antibodies are used that recognize and bind the PINCH-1 protein in a highly specific manner. After the antibodies have been given to the tumor cells, the tumor cells are treated with radiation and/or chemotherapy. The binding of the antibodies to the PINCH-1 protein blocks its function and leads to an increased sensitivity of the tumor cell relative to radiation therapy or chemotherapy. The survival rate of the tumor cells after the treatment with radiation therapy or chemotherapy is thereby reduced significantly.

In this connection, monoclonal anti-PINCH-1-antibodieS, in particular humanized anti-PINCH-1-antibodies, are used preferably. A humanized monoclonal antibody comprises the PINCH-1-binding hypervariable regions of the inventive monoclonal antibody and the framework regions of the variable and constant regions of the light and heavy chains of a human antibody. Methods for producing humanized antibodies are known and inter alia disclosed in Morrison et al. (1984), Jones et al. (1986), Verhoeyen et al. (1988), Riechmann et al. (1988), Queen et al. (1989), and Tempest (1991).

Antibodies in the inventive sense moreover are to be understood as different modified forms, for example, fragments like the Fv fragment, the Fab fragment, the (Fab)′2 fragment or single chain antibodies (gene-technologically produced bi-specific antibodies that are comprised only of two binding domains that are linked by short links). Methods for producing F(ab₂) or F(ab) fragments are known and described in Current Protocols in Immunology (John Wiley & Sons, http://www.wiley.com/legacy/cp/cpi/).

Inventive antibodies are, for example, a monoclonal mouse-anti-PINCH-1-antibody of the IgG1 isotype (clone PINCH-C58; Sigma-Aldrich, DE), a monoclonal mouse-anti-PINCH-1-antibody of the IgM-isotype (clone PINCH-N173; Sigma-Aldrich, DE) and a monoclonal mouse-anti-PINCH-antibody of the IgG2a isotype (clone 49; Becton Dickinson, DE).

In another embodiment of the invention the expression of the PINCH-1-encoding gene is suppressed by the use of RNA interference (RNAi). RNA interference is understood as a mechanism with which by use of target-recognizing RNA molecules the expression of a gene is inhibited.

In a preferred embodiment, the molecules that suppress the expression of the PINCH-1 protein by RNA interference are the so-called siRNA (small interfering RNA) molecules. siRNA molecules are short single-strand or double-strand RNA molecules which can inhibit the expression of a target gene specifically. By administering PINCH-1-siRNA it is prevented that the tumor cell expresses the PINCH-1 gene and thus forms the PINCH-1 protein. This also leads to a raised sensitivity of the tumor cell relative to radiation therapy or chemotherapy.

Preferred siRNAs of the invention are oligo nucleotides of from 18 to 30 nucleotides, preferred from 21 to 23 nucleotides which are comprised either of a single RNA strand that is homolog to a partial sequence of the human PINCH-1-cDNA (SEQ ID No. 1, Genbank Accession Number NM_{—}004987) or is comprised of a double stranded RNA, of which one strand is homolog to a partial sequence of the PINCH-1-cDNA (SEQ ID No. 1) and the other is complementary to the first strand. Homolog means in this context identity between two sequences of at least 80%, preferably more than 90%, particularly preferred more than 95%.

The PINCH-1-cDNA is that DNA strand that is obtained when one translates mature mRNA transcribed by the PINCH-1 gene by the enzyme reverse transcriptase into DNA strand that is complementary to this DNA strand.

Preferably, to the 3′ end of the siRNA additionally two deoxythymidine residues are added.

Human PINCH-1-cDNA (SEQ ID No. 1)

| | |
|---|---|
| 1 | tagttcaaga caacagagac aaagctaaga tgaggaagtt ctgtacagtt taggaaatag |
| 61 | aggctttcaa agataattcg cagtgatgtg aaactggcct cccaagccct gataacaaca |
| 121 | tggccaacgc cctggccagc gccacttgcg agcgctgcaa gggcggcttt gcgcccgctg |
| 181 | agaagatcgt gaacagtaat ggggagctgt accatgagca gtgtttcgtg tgcgctcagt |
| 241 | gcttccagca gttcccagaa ggactcttct atgagtttga aggaagaaag tactgtgaac |
| 301 | atgactttca gatgctcttt gccccttgct gtcatcagtg tggtgaattc atcattggcc |
| 361 | gagttatcaa agccatgaat aacagctggc atccggagtg cttccgctgt gacctctgcc |
| 421 | aggaagttct ggcagatatc gggtttgtca agaatgctgg gagacacctg tgtcgcccct |
| 481 | gtcataatcg tgagaaagcc agaggccttg ggaaatacat ctgccagaaa tgccatgcta |
| 541 | tcatcgatga gcagcctctg atattcaaga acgaccccta ccatccagac catttcaact |
| 601 | gcgccaactg cgggaaggag ctgactgccg atgcacggga gctgaaaggg gagctatact |
| 661 | gcctcccatg ccatgataaa atgggggtcc ccatctgtgg tgcttgccga cggcccatcg |
| 721 | aagggcgcgt ggtgaacgct atgggcaagc agtggcatgt ggagcatttt gtttgtgcca |
| 781 | agtgtgagaa accctttctt ggacatcgcc attatgagag gaaaggcctg gcatattgtg |
| 841 | aaactcacta taaccagcta tttggtgatg tttgcttcca ctgcaatcgt gttatagaag |
| 901 | gtgatgtggt ctctgctctt aataaggcct ggtgcgtgaa ctgctttgcc tgttctacct |
| 961 | gcaacactaa attaacactc aagaataagt ttgtggagtt tgacatgaag ccagtctgta |
| 1021 | agaagtgcta tgagaaattt ccattggagc tgaagaaaag acttaagaaa ctagctgaga |
| 1081 | ccttaggaag gaaataagtt cctttatttt ttcttttcta tgcaagataa gagattacca |
| 1141 | acattacttg tcttgatcta cccatattta aagctatatc tcaaagcagt tgagagaaga |
| 1201 | ggacctatat gaatggtttt atgtcatttt tttaattaaa aaagaaaaat tcatataatc |
| 1261 | gtgtttaaaa cacaaatgaa gtcagtattt gcctttgtta acccttatcc atttgttgac |
| 1321 | atgtagactg tttacaaaaa aaaaacacat ggttaaatgt taaattttaa ttaaggcccc |
| 1381 | caaaaattaa atataacttt ttaaaatgaa aggagtcacc ttttacatga ctcaggtgaa |
| 1441 | aaaacagtat aaacattaat ttactttgtg ttcaaaagaa aattccaact gctgttgggg |
| 1501 | aaggacacag aaaagaaaaa taaccaccca aaaaaaaaaa aaaaaaaaaa aaaaaaaaa |

PINCH-1-cDNA is to be understood to include all sequences which have with the sequence SEQ ID No.1 a match of 95% or more, preferably 98%. The preparation of siRNA is carried out in accordance with a method known to a person skilled in the art.

Inventive examples of PINCH-1-siRNA are, for example, the poly nucleotides according to SEQ ID No. 2, SEQ ID No. 3, SEQ ID No. 4 and SEQ ID No. 5, as well as variations (homologs) that differ with respect to the sequence slightly, but still block the function of the PINCH-1 protein. Preferably, homolog variations of the poly nucleotides according to SEQ ID No. 2 to 5 differ relative to them in at most from 1 to 4 nucleotides, preferably by not more than one or two nucleotides.

| | |
|---|---|
| 1) GGACCUAUAUGAAUGGUUUtt | (SEQ ID No. 2) |
| 2) GGACUCUUCUAUGAGUUUGtt | (SEQ ID No. 3) |
| 3) GGAAGAAAGUACUGUGAACtt | (SEQ ID No. 4) |
| 4) GCUAUAUCUCAAAGCAGUUtt | (SEQ ID No. 5) |

In this connection, the invention also encompasses complementary sequences with modified backbone. The term nucleotide sequences with modified backbone encompasses all other linear polymers in which the bases adenine (A), cytosine (C), guanine (G) and uracil (U) or thymine (T) are arranged in suitable sequence, for example, sequences with a phosphothioate, phosphoamidate or O-methyl-derivatized backbone, peptide nucleic acids (PNA), locked nucleic acids (LNA), nucleic acids with mixed backbone or morpholinos as well as fluorochrome (green/red/etc. fluorescence protein) marked sequences, and that can inhibit the expression of the PINCH-1 gene can restrain.

After transfection of siRNA (i.e. the introduction of specific RNA molecules into eukaryotic cells) the tumor cells are treated in vitro with radiation and/or cytostatic agents (chemotherapy). As a radiation source a conventional 200-kV-X-ray tube (13 mA, ˜1,3 Gy/min.) can be used advantageously.

As cytostatic agents, for example, cisplatin (Platinex®, Bristol-Myers-Squibb, Munich) and Mitomycin C (Mitomycin medac®, Gesellschaft für klinische Spezialpräparate mbH, Wedel) are used.

With the aid of attached Figures, embodiments of the invention are explained in more detail.

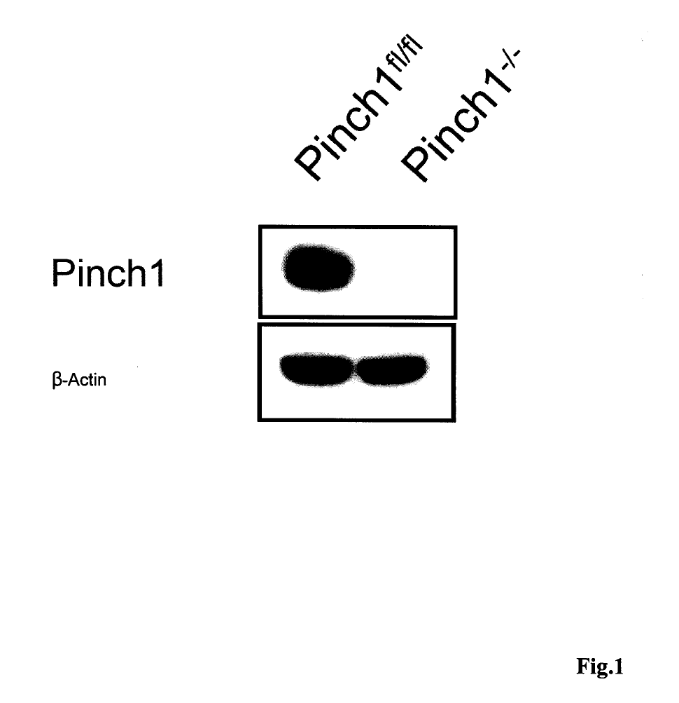

FIG. 1 Western Blot

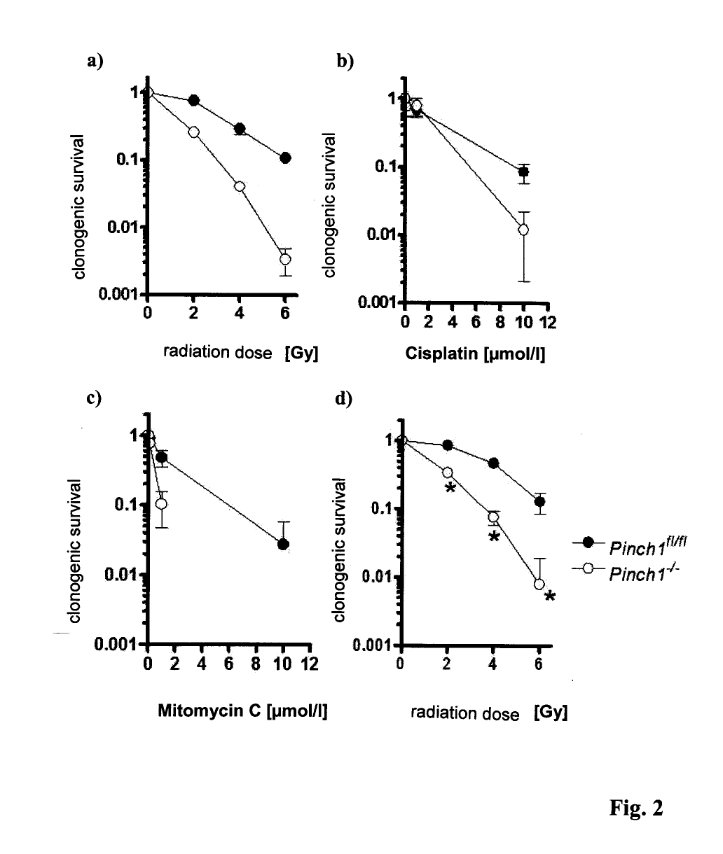

FIG. 2 treatment diagrams

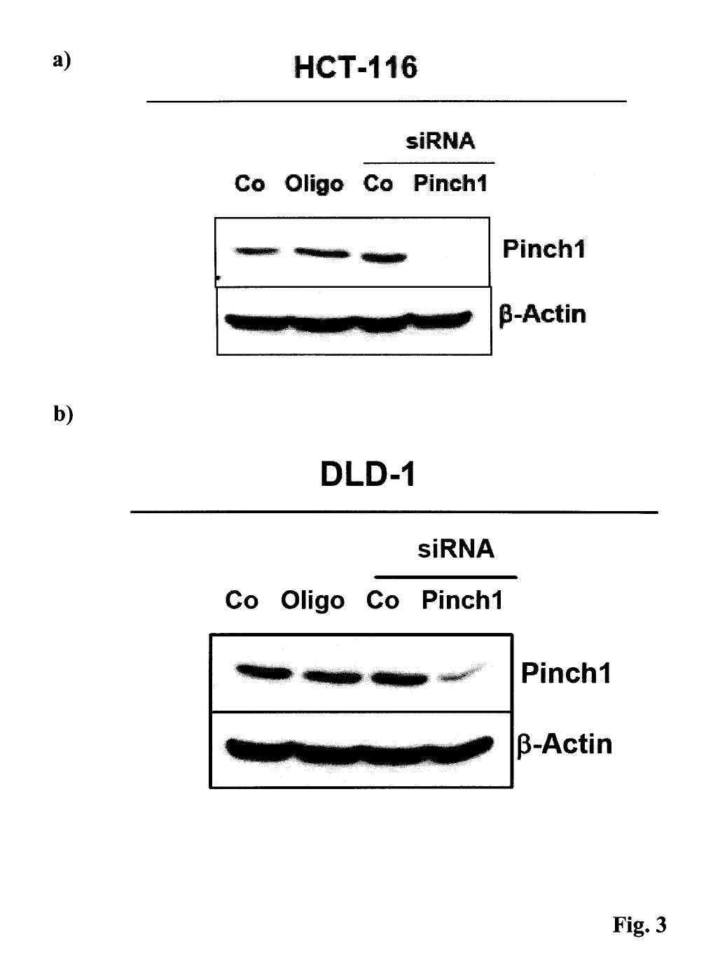

FIG. 3 Western Blot

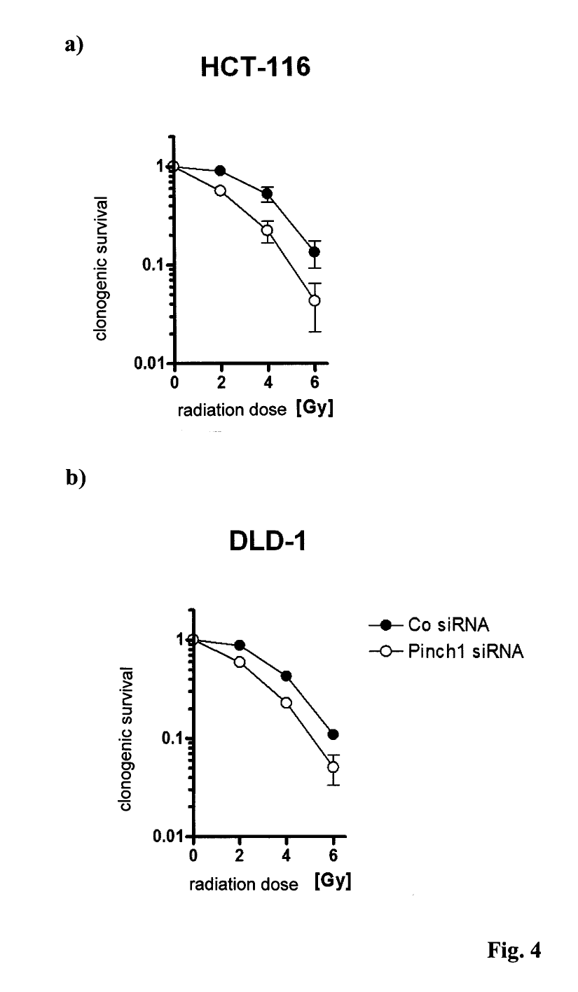

FIG. 4 treatment diagrams

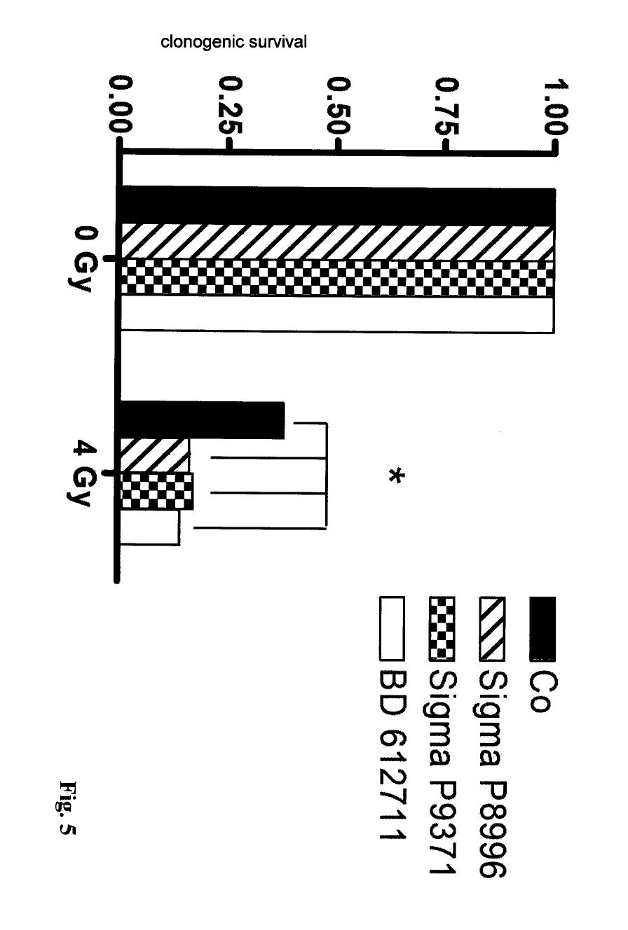

FIG. 5 treatment diagrams

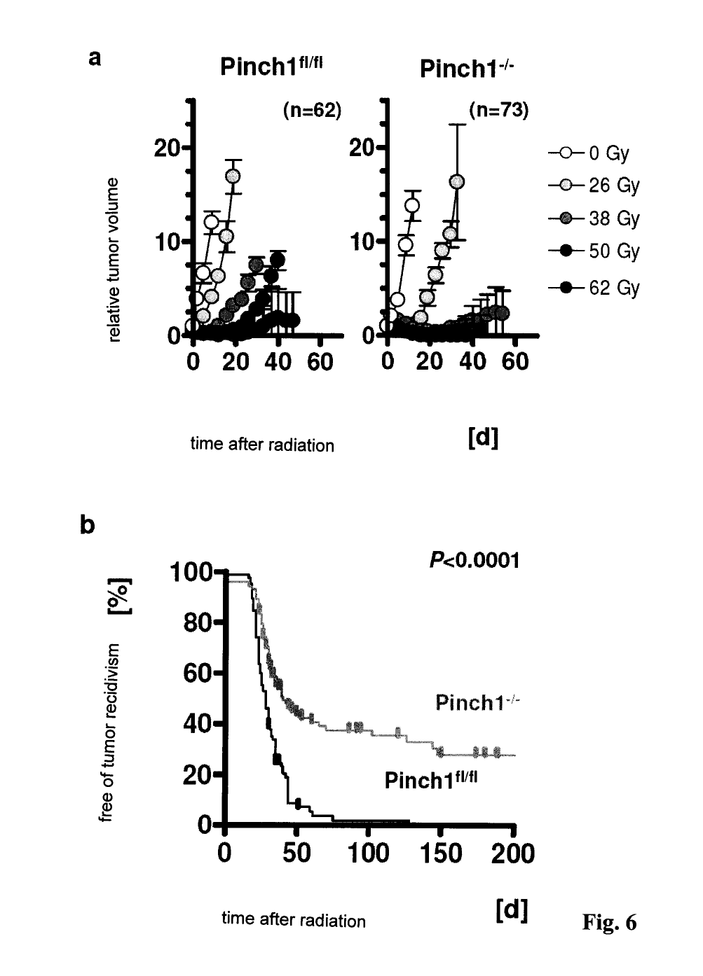

FIG. 6 tumor growth and recurrence of tumors after radiation

### EMBODIMENT 1

In immortalized embryonic mouse fibroblasts the gene encoding for PINCH-1 was switched off. A standard method for producing transgenic mice is disclosed, for example, in WO/1999/036528.

By genetically switching off the PINCH-1 gene the cells became clearly more sensitive relative to treatment with X-rays or cytostatic agents, i.e. the survival rate of these cells after radiation or chemotherapy was lowered significantly compared with PINCH-1 expressing control cells. Cells were disposed in two-dimensional or three-dimensional cell culture models and treated with X-ray radiation (200 kV, 13 mA, ˜1,3 Gy/min., 0-10 Gy) or the cytostatic agent cisplatin (Platinex®) or mitomycin C (Mitomycin medac®) in a concentration of 0-10 μmol/l (1H or 24 h). The clonogenic cell survival, i.e. the proliferative integrity of the treated cells, was measured. The grown cell colonies were fixed, stained and the colony number was determined microscopically.

FIG. 1 shows a Western Blot which proves that in the mouse cells in which the PINCH-1-encoding gene has been switched off no PINCH-1 protein is formed (on the right), in contrast to control cells in which PINCH-1 protein is present (on the left).

FIG. 2 shows the clonogenic survival rate of the mouse cells in which the PINCH-1-encoding gene has been switched off as well as wild-type control cells, after radiation (FIGS. 2a, d) as well as after treatment with the cytostatic agent cisplatin (FIG. 2b), mitomycin C (FIG. 2c) in two-dimensional (FIGS. 2a, b, c) and three-dimensional (FIG. 2d) cell cultures.

### EMBODIMENT 2

Human colorectal tumor cell lines of the type HCT-116 (ATCC No. CCL-247) and of the type DLD-1 (ATCC No. CCL-221) were transfected with PINCH-1-siRNA of the SEQ ID No. 5. For this purpose, 3×10⁵ tumor cells on 6-well plates (BD, Heidelberg) in Dulbecco's Modified Eagle (DMEM; Gibco, Karlsruhe) with Glutamax-I (L-alanyl-L-glutamine), 10% serum (FCS; Biochrom, Berlin) and 1% of non-essential amino acids (Gibco, Karlsruhe) were seeded and cultured at 37° C. and 7% CO₂. After 24 h the cells were washed once with Opti-MEM I (Invitrogen, Karlsruhe) and transfected with oligo fectamine (0.2%) and 20 nmol/l PINCH-1-siRNA for 8 h without serum in Opti-MEM I. Afterwards 10% serum was added and the cells incubated for another 16 h at 37° C. and 7% CO₂. After a total of 24 h after transfection the cells were washed with phosphate-buffered saline solution (PBS; PAA, Cölbe) and removed with trypsin/EDTA solution. A portion of the cells was used for determining the clonogenic survival after radiation (colony forming assay), while another portion was cultured for another 24 h. Of this portion, protein lysates were produced for examining knock-downs in the Western Blot, the proteins were separated by means of SDS PAGE, transferred onto a nitrocellulose membrane and detected with a mouse-anti-PINCH-1-antibody (clone 49; BD Heidelberg) as well as a peroxidase-coupled goat-anti-mouse-secondary antibody and the ECL system (GE Healthcare, Munich). As shown in FIG. 3, the Western Blot shows that the formation of PINCH-1 protein by aforementioned specific siRNA sequence has indeed been prevented. Treatment of the cells with unspecific control-siRNA (Co) or with the transfection reagent (oligo, oligo fectamine) alone showed no change of the PINCH-1 expression. FIG. 4 shows that after radiation the survival rate is lowered significantly in the tumor cells when treated with PINCH-1-siRNA in comparison to the cells with control-siRNA (co or co siRNA). siRNA was transfected in a concentration of 20 nmol/1 and the cells were irradiated 48 h later with Gy 0-6.

### EMBODIMENT 3

By blocking the PINCH-1 protein by binding a specific anti-PINCH-1-antibody, the sensitivity of tumor cells can be increased relative to radiation or chemotherapy. In this connection, for determining clonogenic survival after treatment with an anti-PINCH-1-antibody cells were seeded on 24-well plates (BD Heidelberg) in Dulbecco's Modified Eagle medium (DMEM; Gibco, Karlsruhe) with Glutamax-I (L-alanyl-L-glutamine), 10% serum (FCS; Biochrom, Berlin) and 1% non-essential amino acids (Gibco, Karlsruhe). After culturing at 37° C. and 7% CO₂ for 24 h, 50 μg/ml of anti-PINCH-1-antibody and unspecific control antibody (Santa Cruz, Heidelberg) were added followed by incubation for another 24 h. Then radiation at 200 kV X-ray radiation (Yxlon Y.TU 320, Yxlon, Copenhagen, Denmark; 20 mA, ˜1,3 Gy/min.) was performed. Eight days after seeding the cells were fixed with 80% ethanol for 30 min., were stained with Coomassie Blue solution, and colonies with more than 50 cells were counted.

FIG. 5 shows cell survival of human colorectal tumor cell lines (HCT-116, DLD-1) that had been treated with anti-PINCH-1-antibodies (clone PINCH-C58 (Sigma P8896), clone PINCH-N173 (Sigma P9371), clone 49 (Beckton-Dickinson catalogue No. 612711) or unspecific control antibody (co) 24 h before radiation (0 or 4 Gy). The tumor cells were significantly sensitized by the treatment with the specific PINCH 1 antibodies relative to radiation in comparison to the control cells.

### EMBODIMENT 4

To verify the radiation sensitivity of Pinch1^{fl/fl} (Pinch1^{floxed/floxed})—relative to Pinch1^{-/-} tumor cells in vivo, allograft tumors were generated in immune-suppressed NMRI (nu/nu) naked mice. The tumors were characterized by their response to radiation, their growth rate, their proliferation, their oxygen supply (hypoxia), their supply through blood vessels and their blood circulation. The response of the tumors to radiation was determined by the tumor volume as well as the time up to the local recurrence of tumors.

Male and female immuno-suppressed NMRI mice (genotype nu/nu; absence of the thymus and the hair) of 7 to 14 weeks of age (Experimentelles Zentrum der Medizinischen Fakultät, Technische Universität Dresden) were further immuno-suppressed from 1 to 5 days before tumor grafting by whole body radiation (1×4 Gy, 200 kV X-rays, 0.5 mm copper filter, 1 Gy/min.). The animal cages were equipped with a 12-hour daylight/darkness cycle (the light was switched on at 7 a.m., respectively,), a constant temperature of 26° C. and a relative humidity of 50-60%. The mice were fed a commercially available diet for lab animals and optionally water. For producing the allograft tumors in vivo the immortalized Pinch1^{-/-} and Pinch1^{fl/fl} mouse fibroblasts were injected subcutaneously into the axilla of recipient mice. After knots of palpatable size had formed, the tumors were cut out and transplanted subcutaneously in a group by animals. Tumors with an average growth rate were cut out from them, were divided into pieces of about 1 mm size, placed into a medium and stored for other experiments in liquid nitrogen.

For the experiments the obtained tumor pieces were transplanted on the back of 5 mice (first pass). After tumors with a diameter of 10 to 15 mm had formed, the tumor with average growth rate was cut out and pieces of about 1 mm size were transplanted on the backs of 10 further animals (second and third passes). For the experiments tumors of the second and third passes with an average growth rate were cut out and pieces of about 1 mm size were transplanted subcutaneously in the right hind leg. The cut out tumors were characterized in that they were subjected to a DNA or protein isolation. The presence of the Pinch1 knockout in Pinch1^{-/-} tumors or the Pinch1^{floxed} sequence in Pinch1^{fl/fl} tumors was checked by PCR. The expression of PINCH1-protein was checked by Western Blot.

The local radiation of the tumors was carried out at 200 kV X-rays (0.5 mm copper filter, radiation dose 1 Gy/min., Röntgenwerke Seifert) on the right hind leg. After the tumor had reached a volume of 0.10 to 0.32 cm³, the animals were divided randomly into groups of four animals for the different radiation doses. Single doses of 26, 32, 38, 44, 50, 56 or 62 Gy were applied 2 minutes later with locally cut-off blood flow of the thigh of the tumor-bearing leg of anesthetized mice (120 mg/kg of body weight ketamine i.p. (intraperitoneal) and 16 mg/kg xylazine i.p.). Pinch1^{fl/fl} and Pinch1^{-/-} tumors were evenly distributed across the different dosage groups.

The diameter of the tumors was determined twice weekly with a sliding caliper. The tumor volume was calculated by the formula for a rotational ellipsoid (π/6·a·b²) wherein a is the longer and b is the shorter tumor axis at a right angle thereto. The growth duration (tumor growth time, TGT) of unirradiated and irradiated Pinch1^{fl/fl} and Pinch1^{-/-} tumors was determined directly from the growth curves of individual tumors at that time that was required after the start of the experiment to reach 2-(TGTᵥ₂) or 5-fold (TGTᵥ₅) of the initial volume. Average values for the growth duration from Pinch1^{fl/fl} and Pinch1^{-/-} tumors were compared by means of the Mann-Whitney U-test (GraphPad Prism software 4.03). The tumor volume was measured until the tumors reached a volume of approx. 1.5 cm³. The frequency of the recurrence of tumors after radiation was determined when the tumor volume increased again for three successive measurements after reaching a lowest point.

All together the local tumor control (is reciprocal to the rate of regrowth of tumors) of 85 Pinch1^{fl/fl} and 99 Pinch1^{-/-} tumors was evaluated. In the animals with Pinch1^{fl/fl} and Pinch1^{-/-} tumors all reappearing tumors up to the day 210 after radiation were recorded. It has been found that all Pinch1^{-/-} tumors for a single dose of 38 Gy were locally controlled. For Pinch1^{fl/fl} tumors a local control did not result for any of the applied radiation doses. Mathematical estimations for the time up to the local recurrence of tumors were obtained by means of the Kaplan-Meier method and compared by means of the log rank test (GraphPad Prism software 4.03).

The tumors were irradiated with increasing single doses of X-ray radiation (26-62 Gy) and were observed up to 210 days afterwards. In agreement with in vitro results Pinch1^{-/-} allografts in comparison to Pinch1^{fl/fl} allografts showed a substantially higher radiation sensitivity, in regard to the delay of the tumor growth as well as the survival without recurrence of new tumors. FIG. 6a shows a plot of the tumor volume over time after radiation, FIG. 6B the Kaplan-Meier analysis, i.e. the survival without recurrence of new tumors for subcutaneously growing Pinch1^{fl/fl} and Pinch1^{-/-} allograft tumors in immuno-suppressed mice. In case of the tumor volume (FIG. 6a) each data point stands for the average value±standard error of from 10 to 18 mice. With regard to the S phase marker BrdU, the vascularization, the oxygen supply and necrosis (tested by immune-histochemical methods; data not shown) no significant differences could be determined between Pinch1^{fl/fl} and Pinch1^{-/-} tumors. PINCH-1 plays thus a crucial role for the cell survival in case of radiation and works regardless of the extra-cellular matrix, of in vitro and in vivo growth conditions terms, and of the microenvironment of the tumor.

What is claimed is:

1.-7. (canceled)

8. A method for sensitizing tumor cells relative to radiation and/or chemotherapy, the method comprising the step of applying to the tumor cells an effective amount of a substance that blocks or limits the function of the PINCH-1 protein.

9. The method according to claim 8, wherein the substance is an anti-PINCH-1-antibody.

10. The method according to claim 9, wherein the anti-PINCH-1-antibody is a monoclonal mouse-anti-PINCH-1-antibody of the IgG1 subtype named clone PINCH-C58 (Sigma-Aldrich, DE), a monoclonal mouse-anti-PINCH-1-antibody of the IgM subtype named clone PINCH-N173 (Sigma-Aldrich, DE), or a monoclonal mouse-anti-PINCH-antibody of the IgG2a subtype named clone 49 (Becton Dickinson, DE).

11. The method according to claim 8, wherein the substance inhibits the expression of the PINCH-1 gene.

12. The method according to claim 11, wherein the substance inhibits the expression of the PINCH-1 gene post-transcriptionally.

13. The method according to claim 12, wherein the substance is PINCH-1-siRNA and wherein the siRNA either
a. is comprised of a single-strand RNA that is homolog to a partial sequence of the human PINCH-1 gene (SEQ ID No. 1),
or
b. is comprised of a RNA double strand of which the first strand is homolog to a partial sequence of the PINCH-1 gene (SEQ ID No.1) and the second strand is complementary to the first strand.

14. The method according to claim 13, wherein the PINCH-1-siRNA is at least one sequence selected from the group of sequences consisting of SEQ ID No. 2, SEQ ID No. 3, SEQ ID No. 4, and SEQ ID No. 5.

15. A method for sensitizing tumor cells relative to radiation and/or chemotherapy, the method comprising the step of administering to a living being an effective amount of a substance that blocks or limits the function of the PINCH-1 protein.

16. The method according to claim 15, wherein the substance is an anti-PINCH-1-antibody.

17. The method according to claim 16, wherein the anti-PINCH-1-antibody is a monoclonal mouse-anti-PINCH-1-antibody of the IgG1 subtype named clone PINCH-C58 (Sigma-Aldrich, DE), a monoclonal mouse-anti-PINCH-1-antibody of the IgM subtype named clone PINCH-N173 (Sigma-Aldrich, DE), or a monoclonal mouse-anti-PINCH-antibody of the IgG2a subtype named clone 49 (Becton Dickinson, DE).

18. The method according to claim 15, wherein the substance inhibits the expression of the PINCH-1 gene.

19. The method according to claim 18, wherein the substance inhibits the expression of the PINCH-1 gene post-transcriptionally.

20. The method according to claim 19, wherein the substance is PINCH-1-siRNA and wherein the siRNA either
a. is comprised of a single-strand RNA that is homolog to a partial sequence of the human PINCH-1 gene (SEQ ID No. 1),
or
b. is comprised of a RNA double strand of which the first strand is homolog to a partial sequence of the PINCH-1 gene (SEQ ID No.1) and the second strand is complementary to the first strand.

21. The method according to claim 20, wherein the PINCH-1-siRNA is at least one sequence selected from the group of sequences consisting of SEQ ID No. 2, SEQ ID No. 3, SEQ ID No. 4, and SEQ ID No. 5.


SEQUENCE LISTING (5 sequences)

SEQ ID NO: 1 - DNA, 1559 bp, Homo sapiens
tagttcaagacaacagagacaaagctaagatgaggaagttctgtacagtttaggaaatagaggctttcaaagataattcgcagtgatgtgaaactggcctcccaagccctgataacaacatggccaacgccctggccagcgccacttgcgagcgctgcaagggcggctttgcgcccgctgagaagatcgtgaacagtaatggggagctgtaccatgagcagtgtttcgtgtgcgctcagtgcttccagcagttcccagaaggactcttctatgagtttgaaggaagaaagtactgtgaacatgactttcagatgctctttgccccttgctgtcatcagtgtggtgaattcatcattggccgagttatcaaagccatgaataacagctggcatccggagtgcttccgctgtgacctctgccaggaagttctggcagatatcgggtttgtcaagaatgctgggagacacctgtgtcgcccctgtcataatcgtgagaaagccagaggccttgggaaatacatctgccagaaatgccatgctatcatcgatgagcagcctctgatattcaagaacgacccctaccatccagaccatttcaactgcgccaactgcgggaaggagctgactgccgatgcacgggagctgaaaggggagctatactgcctcccatgccatgataaaatgggggtccccatctgtggtgcttgccgacggcccatcgaagggcgcgtggtgaacgctatgggcaagcagtggcatgtggagcattttgtttgtgccaagtgtgagaaaccctttcttggacatcgccattatgagaggaaaggcctggcatattgtgaaactcactataaccagctatttggtgatgtttgcttccactgcaatcgtgttatagaaggtgatgtggtctctgctcttaataaggcctggtgcgtgaactgctttgcctgttctacctgcaacactaaattaacactcaagaataagtttgtggagtttgacatgaagccagtctgtaagaagtgctatgagaaatttccattggagctgaagaaaagacttaagaaactagctgagaccttaggaaggaaataagttcctttattttttcttttctatgcaagataagagattaccaacattacttgtcttgatctacccatatttaaagctatatctcaaagcagttgagagaagaggacctatatgaatggttttatgtcatttttttaattaaaaaagaaaaattcatataatcgtgtttaaaacacaaatgaagtcagtatttgcctttgttaacccttatccatttgttgacatgtagactgtttacaaaaaaaaaacacatggttaaatgttaaattttaattaaggcccccaaaaattaaatataactttttaaaatgaaaggagtcaccttttacatgactcaggtgaaaaaacagtataaacattaatttactttgtgttcaaaagaaaattccaactgctgttggggaaggacacagaaaagaaaaataaccacccaaaaaaaaaaaaaaaaaaaaaaaaaaaaaa

SEQ ID NO: 2 - DNA, 21 bp, Artificial Sequence
  siRNA
ggaccuauaugaaugguuutt

SEQ ID NO: 3 - DNA, 21 bp, Artificial Sequence
  siRNA
ggacucuucuaugaguuugtt

SEQ ID NO: 4 - DNA, 21 bp, Artificial Sequence
  siRNA
ggaagaaaguacugugaactt

SEQ ID NO: 5 - DNA, 21 bp, Artificial Sequence
  siRNA
gcuauaucucaaagcaguutt